(19) United States
(12) Patent Application Publication
(10) Pub. No.: US 2007/0190137 A1
(43) Pub. Date: Aug. 16, 2007

(54) Osmotic dosage form with controlled release and fast release aspects

(76) Inventors: Reyes Iran, San Jose, CA (US); Julie Lee, Sunnyvale, CA (US); Brian L. Barclay, Sunnyvale, CA (US); Nipun Davar, Fremont, CA (US); Ramkumar Subramanian, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/528,053

(22) Filed: Sep. 26, 2006

Related U.S. Application Data
(60) Provisional application No. 60/724,470, filed on Oct. 7, 2005.

(51) Int. Cl. A61K 9/24

(52) U.S. Cl. 424/465

(57) ABSTRACT

Disclosed are osmotic dosage forms including a semi-permeable membrane; a first and a second orifice in the semi-permeable membrane located at opposite ends of the semi-permeable membrane; a controlled release drug layer located adjacent to the first orifice and within the semi-permeable membrane; a fast release drug layer located adjacent to the second orifice and within the semi-permeable membrane; a push layer located within the semi-permeable membrane and between the controlled release drug layer and the fast release drug layer; a barrier layer slidably located between the push layer and the fast release drug layer; and wherein an area of the second orifice is greater than or equal to about 7800 mil². Also disclosed are methods of making and using such osmotic dosage forms.

### CROSS REFERENCE TO RELATED U.S. APPLICATION DATA

The present application is derived from and claims priority to provisional application U.S. Ser. No. 60/724,470, filed Oct. 7, 2005, which is herein incorporated by reference in its entirety.

### FIELD OF THE INVENTION

The invention pertains to osmotic dosage forms having a controlled release drug layer and a fast release drug layer, and related methods.

### BACKGROUND

Osmotic dosage forms for controlled delivery of drugs have been known in the art for a number of years. In certain circumstances, it is desirable to combine fast release of a drug together with controlled release of a same or different drug from the same dosage form.

For instance, U.S. Pat. Nos. 4,814,181, 4,915,953, 5,240,713, and 4,915,954 describe osmotic controlled release dosage forms that comprise a first lamina that is delivered in a short period of time and a second lamina that is delivered in a prolonged period of time. However, these osmotic dosage forms do not provide for a push layer that can promote better delivery control.

U.S. Pat. Nos. 6,919,373 and 6,930,129 disclose use of an immediate release overcoat for the delivery of methylphenidate from osmotic dosage forms. However, overcoating an osmotic dosage form may add cost, and reduce delivery control of the immediate release component.

U.S. Pat. Nos. 6,387,403, and 6,630,165 disclose use of a barrier layer in an osmotic dosage form, for the purpose of reducing mixing during operation of active agent between layers in the osmotic dosage form. No mention is made of how to obtain fast release of a drug from the osmotic dosage forms.

Accordingly, there remains a need for dosage forms and methods that combine fast release of a drug together with controlled release of a same or different drug from the same dosage form.

### SUMMARY OF THE INVENTION

In an aspect, the invention relates to an osmotic dosage form comprising: a semi-permeable membrane; a first and a second orifice in the semi-permeable membrane located at opposite ends of the semi-permeable membrane; a controlled release drug layer located adjacent to the first orifice and within the semi-permeable membrane; a fast release drug layer located adjacent to the second orifice and within the semi-permeable membrane; a push layer located within the semi-permeable membrane and between the controlled release drug layer and the fast release drug layer; a barrier layer slidably located between the push layer and the fast release drug layer; and wherein an area of the second orifice is greater than or equal to about 7800 mil².

In another aspect, the invention relates to a method of making an osmotic dosage form comprising: providing a semi-permeable membrane; locating a first and a second orifice in the semi-permeable membrane at opposite ends of the semi-permeable membrane; locating a controlled release drug layer adjacent to the first orifice and within the semi-permeable membrane; locating a fast release drug layer adjacent to the second orifice and within the semi-permeable membrane; locating a push layer within the semi-permeable membrane and between the controlled release drug layer and the fast release drug layer; slidably locating a barrier layer between the push layer and the fast release layer; and wherein an area of the second orifice is greater than or equal to about 7800 mil².

### BRIEF DESCRIPTION OF THE DRAWINGS

FIGS. 1A-C show an osmotic dosage form according to the invention.

FIG. 2 shows another osmotic dosage form according to the invention.

FIG. 3 shows in vitro release rates for a dosage form according to the invention.

FIGS. 4-11 show results from the experimental series of Example 2.

### DETAILED DESCRIPTION

I. Introduction

The inventors have unexpectedly discovered that it is possible to address the problems noted above in the art using an osmotic dosage form comprising: a semi-permeable membrane defining a capsule shape; a first and a second orifice in the semi-permeable membrane located at opposite ends of the semi-permeable membrane; a controlled release drug layer located adjacent to the first orifice and within the semi-permeable membrane; a fast release drug layer located adjacent to the second orifice and within the semi-permeable membrane; a push layer located within the semi-permeable membrane and between the controlled release drug layer and the fast release drug layer; a barrier layer slidably located between the push layer and the fast release drug layer; wherein an area of the second orifice is greater than or equal to about 7800 mil².

In particular, slidably locating the barrier layer allows it to move within the dosage form once the dosage form is in operation. Consequently the barrier layer provides an anchor or base against which the push layer can push as the rapid release drug layer is released from the inventive osmotic dosage form. Further, the second orifice size is an important parameter for determining the release rate of the fast release drug layer. Thus, the release rate through the second orifice can be further controlled by varying the size of the orifice. This can be seen in the experimental results presented elsewhere herein.

The invention, and embodiments thereof, will now be described in more detail.

II. Definitions

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Barrier layer” means a layer that is slidably located between the push layer and the fast release layer. In embodiments, the barrier layer may comprise an inert, pharmaceutically acceptable (ie compendial) material, or blend of materials, that posseses little or no water solubility, does not swell appreciably in the presence of water, is resistant to deformation upon compaction, and/or possesses a stiffness sufficient to allow it to withstand the force of the push layer when the barrier layer has slid into place against the first orifice and has blocked it (the barrier layer thus provides a base against which the push layer can push).

Accordingly, the use of water-soluble or highly water-soluble compounds (eg most sugars, sodium salts), and/or highly water-soluble or highly swellable polymers is not advisable. In certain embodiments, stearic acid is blended into the barrier layer material prior to compression to aid in the formation of the compacted barrier layer.

In an embodiment, the barrier layer comprises one or more of ethylcellulose, calcium phosphate dibasic, calcium phosphate tribasic, calcium carbonate, polyvinyl acetate, methylcellulose, cellulose (powder), microcrystalline cellulose, polymethyl methacrylate, talc, and/or combinations of the above, and/or any of the above granulated with PVP or HPMC. In other embodiments, the barrier layer comprises one or more of paraben (butyl, propyl, ethyl, methyl) and/or cellulose mono-acetate and/or combinations of these with other barrier materials.

“Controlled release” or “controllably releasing” means continuous release or continuously releasing of a drug or a dose of a drug over a prolonged period of time.

“Controlled release drug layer” means a drug layer from which the drug is controllably released. In an embodiment, the controlled release drug layer is located adjacent to the first orifice and within the semi-permeable membrane. The materials and methods used to formulate a controlled release drug layer may be chosen conventionally.

“Dosage form” means a drug in a medium, carrier, vehicle, or device suitable for administration to a patient.

“Drug” means a pharmaceutically active agent or a pharmaceutically acceptable salt thereof. Drugs useful in the practice of this invention include, but are not limited to the following: prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, topiramate, paliperidone, oxybutynin, methyl phenidate, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17-varies hydroxyprogesterone acetate, 19-nor-progesterone, norgestrel, norethindrone, norethisterone, norethiederone, progesterone, norgesterone, norethynodrel, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, capropril, mando, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, imipramine, and terazosine HCl di-hydrate. Further examples are proteins and peptides which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, prolactin, somatostatin, lypressin, pancreozymin, luteinizing hormone, LHRH, LHRH agonists and antagonists, leuprolide, interferons, interleukins, growth hormones such as human growth hormone, bovine growth hormone and porcine growth hormone, fertility inhibitors such as the prostaglandins, fertility promoters, growth factors, coagulation factors, human pancreas hormone releasing factor, analogs and derivatives of these compounds, and pharmaceutically acceptable salts of these compounds or their analogs or derivatives, and various combinations of these compounds, and various combinations of these compounds with various pharmaceutically acceptable salts of the above compounds. “Different drugs” means drugs with substantially different chemical structures. “Substantially identical drugs” means drugs with substantially similar chemical structures. “Identical drugs” means drugs with identical chemical structures. In an embodiment, various drugs may be combined within a drug layer.

“Drug layer” means that portion or those portions of a dosage form that comprises an active pharmaceutical ingredient.

“Fast release drug layer” means a drug layer from which the drug is released faster than the controlled release drug layer. In an embodiment, the drug is released at a rate that matches the rate obtained from an immediate release layer overcoated on the osmotic dosage form. In another embodiment, the fast release drug layer releases the drug at a rate that is at least about the rate at which the controlled release drug layer releases the drug, preferably the fast release drug layer releases the drug at a rate that is at least about 1.5 times the rate at which the controlled release drug layer releases the drug, more preferably the fast release drug layer releases the drug at a rate that is at least about 2.0 times the rate at which the controlled release drug layer releases the drug. In an embodiment, the fast release drug layer is located adjacent to the second orifice and within the semi-permeable membrane. In an embodiment, the fast release drug layer comprises a carrier and an optional disintegrant. In embodiments, the carrier may comprise microcrystalline cellulose or Starch 1500. In embodiments, the disintegrant may comprise croscarmellose sodium or cross-linked polyvinylpyrrolidone. The particular carriers and disintegrants used in the fast release layer may be varied, in addition to the amounts of these materials present in the fast release layer, to help in achieving a desired release rate of the fast release layer. The fast release layer may be prepared using conventional techniques for manufacturing osmotic dosage forms, such as granulation and compression or other techniques known in the art.

“Oral” means suitable for oral administration, when used to describe a dosage form.

“Orifice” means a hole or passageway formed through the semi-permeable membrane of an osmotic dosage form. Examples of exit ports and methods of making them that are useful in the practice of this invention are presented elsewhere herein. In an embodiment, an area of the second orifice is greater than or equal to about 7800 mil², preferably greater than or equal to about 10,000 mil², more preferably greater than or equal to about 15,000 mil², even more preferably greater than or equal to about 20,000 mil².

“Osmagent” means a material that establishes an osmotic activity gradient across the semi-permeable membrane. Exemplary osmagents include salts, such as sodium chloride, potassium chloride, lithium chloride, etc. and sugars, such as raffinose, sucrose, glucose, lactose, and carbohydrates.

“Osmotic dosage form” means dosage forms that, in general, utilize osmotic pressure to generate a driving force for imbibing fluid into a compartment formed, at least in part, by a semipermeable membrane that permits free diffusion of fluid but not drug. Examples of osmotic dosage forms useful in the practice of this invention are presented elsewhere herein.

“Patient” means an animal, preferably a mammal, more preferably a human, in need of therapeutic intervention.

“Prolonged period of time” means a continuous period of time of greater than about 2 hours, preferably, greater than about 4 hours, more preferably, greater than about 8 hours, more preferably greater than about 10 hours, more preferably still, greater than about 14 hours, most preferably, greater than about 14 hours and up to about 24 hours.

“Push layer” means a displacement composition that is positioned within the osmotic dosage form such that as the push layer expands during use, the materials forming the controlled release drug layer are expelled from the osmotic dosage form via the first orifice and/or one or more additional orifices located in the semi-permeable membrane adjacent to the first orifice. The push layer can be positioned in contacting layered arrangement with the controlled release drug layer or can have one or more intervening layers separating the push layer and drug layer. In an embodiment, the push layer is located within the semi-permeable membrane and between the controlled release drug layer and the fast release drug layer. The push layer comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

“Semi-permeable Membrane” or “Membrane” means a membrane that is permeable to the passage of an external fluid, such as water and/or biological fluids, but is substantially impermeable to the passage of components such as active pharmaceutical ingredients. Materials useful for forming the semi-permeable membrane are essentially nonerodible and are substantially insoluble in biological fluids during the life of a dosage form that comprises the semi-permeable membrane. Materials and methods for forming the semi-permeable membrane, and structures of osmotic dosage forms that comprise the semi-permeable membrane are disclosed elsewhere herein.

III. Osmotic Dosage Forms According to the Invention

Osmotic dosage forms are known generally in the art. Osmotic dosage forms typically utilize osmotic pressure as a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. Pat: Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

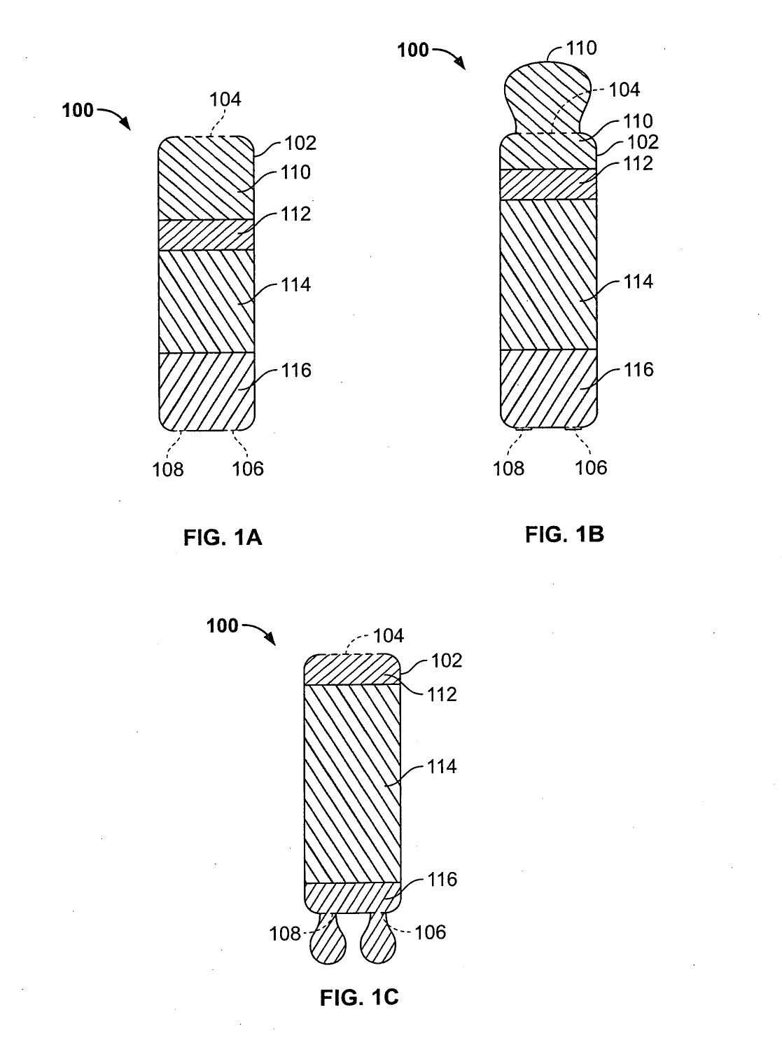

FIGS. 1A-C show an osmotic dosage form according to the invention. Shown is osmotic dosage form 100 which comprises semi-permeable membrane 102, first orifice 104, second orifice 106, additional orifice 108, controlled release drug layer 116, barrier layer 112, push layer 114, and fast release drug layer 110. Located within semi-permeable membrane 102 are controlled release drug layer 116, barrier layer 112, push layer 114, and fast release drug layer 110. First orifice 104 and second orifice 106 are located in semi-permeable membrane 102 at opposite ends of the semi-permeable membrane. Additional orifice 108 is located in semi-permeable membrane 102 adjacent to second orifice 106. Controlled release drug layer 116 is located adjacent to second orifice 106. The materials and methods used to formulate controlled release drug layer 116 may be chosen conventionally. Fast release drug layer 110 is located adjacent to first orifice 104. Push layer 114 is located between controlled release drug layer 116 and barrier layer 112. Barrier layer 112 is slidably located between push layer 114 and fast release drug layer 110.

FIG. 1B shows osmotic dosage form 100 in operation. In operation, fluid from the external environment is absorbed through the semi-permable membrane, and begins to penetrate into controlled release drug layer 116, push layer 114, arid fast release drug layer 110. Fast release drug layer 110 releases quickly from osmotic dosage form 100, before substantial amounts of controlled release drug layer 116 are released from osmotic dosage form 100. As shown in FIG. 1B, barrier layer 112 moves towards first orifice 104 as fast release drug layer 110 is released from osmotic dosage form 100.

Release of fast release drug layer 110 is governed primarily by water uptake directly into the layer and not so much by the water diffusion rate through membrane 102 adjacent to the surface of the fast release drug layer 110. Thus, first orifice 104 size (ie diameter for circular orifices) can play a significant role in governing the ramp-up in release rate through the surface area available for direct water uptake (as opposed to water uptake through semi-permeable membrane 102).

To maximize the ramp-up in release rate from fast release drug layer 110, a maximum first orifice 104 size (e.g. diameter) equaling, or nearly equaling, the relevant dimension (e.g. diameter) of dosage form 100 is desirable. For example, given a 3/16″ (187 mil) diameter longitudinally shaped tablet, release of fast release drug layer 110 should be maximized in instances where first orifice 104 diameter is greater than about 170 mil. As first orifice 104 diameter is reduced to 150 mil, about 76% of the available drug in the rapid-onset layer (for instance containing either microcrystalline cellulose or Starch 1500 as carrier) is released in the first test interval (2-hr duration). Further reduction in first orifice 104 diameter to 110 mil results in 27% of the available drug in fast release drug layer 116 being released, while further reduction yet to 75 mil results in 19% of the available drug being delivered.

Furthermore, for longitudinal tablet embodiments of dosage form 100 of diameter other than 3/16″ (187 mil), maximized release from fast release drug layer 110 should also occur at first orifice 104 diameters approaching the diameter of dosage form 100. For example, for a ¼″ (250 mil) diameter longitudinal tablet dosage form 100, a first orifice 104 diameter of greater that about 230 mil (assuming a circular orifice) should result in the fastest release of drug from fast release drug layer 110). However, it should be noted that, in the case of first orifice 104 diameters (assuming a circular orifice shape) that approach 150 mil, the onset of delivery is similar between longitudinal tablet embodiments of dosage form 100 having a tablet diameter of 3/16″ (187 mil) or ¼″ (250 mil) or, for that matter, 7/32″ (219 mil), suggesting that precise control of onset of delivery is possible over a range of dosage form 100 tablet diameters when first orifice 104 sizes diameters in the region of 150 mil (for a circular orifice) up to the available tablet diameter are applied. Such application allows an approximate 2.5-fold increase in delivery rate during the first release interval relative to the expected steady-state (ie zero order) release rate.

As the hydration rate at the exposed surface of fast release drug layer 110 composition is expected to be similar over dosage form 100 tablet diameter range (for longitudinal tablet shapes; performance of other geometries can be similarly derived), the release rate during the initial test interval relative to the steady-state release rate can be tailored to any other appropriate delivery ratio (defined by Release Rate (initial interval)/Release Rate (steady state)). For example, in an embodiment, for circular first orifice 104 diameters in the region of 110 mil, a 1:1 delivery ratio can be achieved.

FIG. 1C shows osmotic dosage form 100 in further operation. Barrier layer 112 has slid into place against first orifice 104 and has blocked it, thus providing a base against which push layer 114 can push. While this is occurring, push layer 114 has been expanding and driving against controlled release drug layer 116. Drug layer 116 has been absorbing fluid from the external environment through semi-permeable membrane 102 during operation, and has been changing from a solid to a slurry or suspension. Accordingly, as push layer 114 expands, it pushes the slurry or suspension of controlled release drug layer 116 through second orifice 106 and additional orifice 108 and into the environment of use, thus controllably delivering controlled release drug layer 116.

Osmotic dosage forms in accord with the present invention may be manufactured by standard techniques. For example, the osmotic dosage form may be manufactured by the wet granulation technique. In the wet granulation technique, materials making up the fast release drug layer are blended using an organic solvent, such as denatured anhydrous ethanol, as the granulation fluid. Additional ingredients can be dissolved in a portion of the granulation fluid, and this latter prepared solution may be slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for up to 10 minutes. The composition is pressed into a layer, for example, in a Manesty® press or a Korsch multi-layer press. The controlled release drug layer may be prepared in similar fashion. For multi-layer cores according to the invention, the fast release drug layer is pressed into a mold, followed by the barrier layer, the push layer and the controlled release drug layer. These intermediate compression steps typically take place under a force of about 50-100 newtons. Final stage compression typically takes place at a force of 3500 newtons or greater, often 3500-5000 newtons.

Pan coating may be conveniently used to provide the semi-permeable membrane of the completed dosage form. In the pan coating system, the semi-permeable membrane composition is deposited by successive spraying of the appropriate semi-permeable membrane composition onto the compressed multi-layered core, accompanied by tumbling in a rotating pan. A pan coater is used because of its availability at commercial scale. Other techniques can be used for coating the compressed core. Once coated and drilled, the coated cores are dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the semi-permeable membrane of the osmotic dosage form may be formed in one technique using the air-suspension procedure. This procedure consists of suspending and tumbling the compressed core in a current of warmed air and the semi-permeable forming composition, until the semi-permeable membrane is applied to the core. The air-suspension procedure is well suited for independently forming the semi-permeable membrane of the osmotic dosage form. The air-suspension procedure is described in U.S. Pat. No. 2,799,241; in J. Am. Pharm. Assoc., Vol. 48, pp. 451459 (1959); and, ibid., Vol. 49, pp. 82-84 (1960). The osmotic dosage form also can be coated with a Wurster® air-suspension coater using, for example, methylene dichloride blended with methanol as a cosolvent for the semi-permeable membrane forming material. An Aeromatic® air-suspension coater can be used employing a cosolvent.

Additional layers, besides the semi-permeable membrane, may be coated on the osmotic dosage form. Certain additional layers may be known conventionally. For instance, optional water soluble overcoats, which may be colored (e.g., Opadry colored coatings) or clear (e.g., Opadry Clear), may be coated on the osmotic dosage form to provide the finished dosage form.

In an embodiment, the orifices may be drilled in the ends of the osmotic dosage form. In an embodiment, the orifices are formed or formable from a substance or polymer that erodes, dissolves or is leached from the outer wall to thereby form an exit orifice. The substance or polymer may include, for example, an erodible poly(glycolic) acid or poly(lactic) acid in the semipermeable membrane; a gelatinous filament; a water-removable poly(vinyl alcohol); a leachable compound, such as a fluid removable pore-former selected from the group consisting of inorganic and organic salt, oxide and carbohydrate. The orifice, or a plurality of orifices, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice. The exit can have any shape, such as round, triangular, square, elliptical and the like for the uniform metered dose release of a drug from the dosage form. The osmotic dosage form can be constructed with one or more exits in spaced-apart relation or one or more surfaces of the dosage form. Drilling, including mechanical and laser drilling, through the semipermeable membrane can be used to form the orifice(s). Such orifice(s)and equipment for forming such orifice(s)are disclosed in U.S. Pat. No. 3,916,899, by Theeuwes and Higuchi and in U.S. Pat. No. 4,088,864, by Theeuwes, et al.

While there has been described and pointed out features and advantages of the invention, as applied to present embodiments, those skilled in the medical art will appreciate that various modifications, changes, additions, and omissions in the method described in the specification can be made without departing from the spirit of the invention. In particular, it is not intended that the invention be limited in any way to the scope of the following exemplary embodiments.

### IV. EXAMPLES

### Example 1

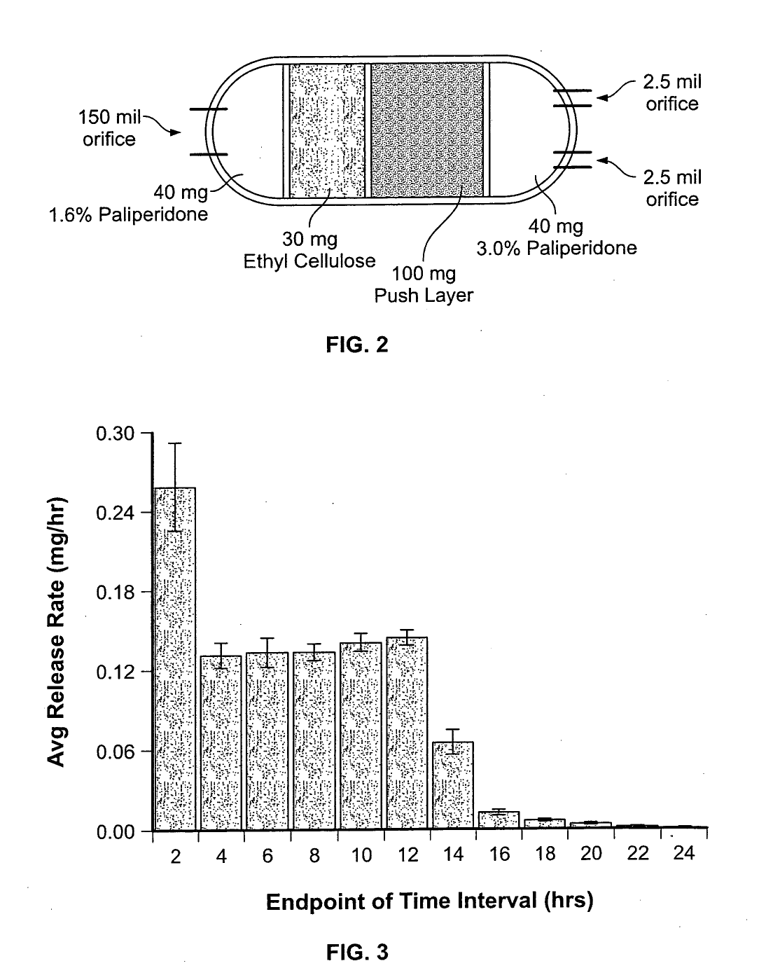

An osmotic dosage form according to the invention that contained a 2 mg dose of paliperidone was formulated as followed. The layer weights for the osmotic dosage form is shown in FIG. 2. The composition of drug layers comprising the core is summarized in Table 1

Beaker scale granulations were prepared and hand-compressed into four-layer systems. A subcoat consisting of hydroxypropyl cellulose:povidone (70:30, % w/w) was applied to a weight gain of 18.6 mg. A membrane coat of cellulose acetate 398-10:PEG 3350 (99:1, % w/w) was applied for an average weight gain of 44.6 mg; the systems were drilled and then dried at about 45° C./45% RH for about 120 hours. Systems release was measured using a Type VII USP dissolution bath. Results are shown in FIG. 3 and Table 2.

TABLE 1

| Material | WG-105 | WG-106 |
|---|---|---|
| Paliperidone | 1.60% | 3.00% |
| Polyethylene oxide N-80 | 0.00% | 71.00% |
| Microcrystalline Cellulose | 67.40% | 0.00% |
| Povidone (K29-32) | 5.00% | 5.00% |
| Stearic Acid | 0.50% | 0.50% |
| Magnesium Stearate | 0.50% | 0.50% |
| Crosspovidone XL | 5.00% | 0.00% |
| Sodium Chloride | 20.00% | 20.00% |

TABLE 2

| Start-up | Residuals | Mass | T₉₀ | | CV |
|---|---|---|---|---|---|
| time | (% of MB) | balance | (hr) | CV within | between |
| 0 hr | 0.48% | 2.0 mg | 12 hr | NA | 3.8% |

### Example 2

The impact of various design variables was tested, including physical variables such as orifice size, weight of the fast release drug layer, and tablet diameter (keeping aspect ratio constant); and formulation variables, such as drug characteristics (mainly solubility), fast release layer diluents, and drug loading.

Table 3 shows the experimental design for Example 2:

TABLE 3
Experimental Design

| | Approximate | Fast Release | |
|---|---|---|---|
| Tablet | Orifice size | Layer weight | Tablet |
| Set # | (mil) | (mg) | diameter |
| 1 | 150 | 40 | ( 3/16″) |
| 2 | 112 | 50 | ( 3/16″) |
| 3 | 75 | 30 | ( 3/16″) |
| 4 | 112 | 30 | ( 3/16″) |
| 5 | 75 | 50 | ( 3/16″) |
| 6 | 150 | 50 | ( 3/16″) |
| 7 | 112 | 40 | ( 3/16″) |
| 8 | 75 | 40 | ( 3/16″) |
| 9 | 150 | 30 | ( 3/16″) |
| 10 | 187.5 | 67 | ( 7/32″) |
| 11 | 187.5 | 100 | (¼″) |
| 12 | 150 | 40 | ( 3/16″) |

The tablets tested were formulated as follows:

Fast release drug layers and controlled release drug layers were formulated using the following recipes:

### Fast Release Drug Layer Formula 1

| Materials | FR 1 |
|---|---|
| Paliperidone | 1.6% |
| Polyox N-80 | 0.0% |
| MCC | 67.4% |
| Povidone | 5.0% |
| Stearic acid | 0.5% |
| Mg Stearate | 0.5% |
| PVP-XL | 5.0% |
| NaCl | 20.0% |

### Controlled Release Drug Layer Formula 1

| Materials | CR 1 |
|---|---|
| Paliperidone | 3% |
| Polyox N-80 | 71% |
| MCC | 0% |
| Povidone | 5% |
| Stearic acid | 0.5% |
| Mg Stearate | 0.5% |
| PVP-XL | 0% |
| NaCl | 20.0% |

### Fast Release Drug Layer Formula 2

| Materials | FR 2 |
|---|---|
| Oxbutynin | 1.6% |
| Polyox N-80 | 0.0% |
| MCC | 87.4% |
| Povidone | 5.0% |
| Stearic acid | 0.5% |
| Mg Stearate | 0.5% |
| PVP-XL | 5.0% |
| NaCl | 0.0% |

### Controlled Release Drug Layer Formula 2

| Materials | CR 2 |
|---|---|
| Oxybutynin | 3% |
| Polyox N-80 | 71% |
| MCC | 0% |
| Povidone | 5% |
| Stearic acid | 0.5% |
| Mg Stearate | 0.5% |
| PVP-XL | 0% |
| NaCl | 20.0% |

Granulation procedure: All the ingredients except stearic acid and magnesium stearate were accurately weighed out into a glass jar. The ingredients were then blended for 10 minutes by placing the jar on a roller mill. The blended mixture was then granulated using an Arrow mixer. Ethanol was added dropwise while mixing and also used as granulating aid. The coarse wet granules were passed through a 20-mesh screen and spread out on a butcher paper. The granules were dried overnight on the counter top under the fume hood. The dried granules were again passed through a 20-mesh screen and transferred to the glass jar. Stearic acid was then added and the mixture was blended for 10 minutes by placing the jar on a roller mill. Magnesium stearate was then added to the granules and blended for 30 seconds by placing the jar on the roller mill.

A push layer was formulated using the following materials, and prepared conventionally:
73.7% Polyethylene Oxide 7000K
20% Sodium Chloride (powder)
5% Povidone K29-32
1% Iron Oxide, Green PB-1581
0.25% Stearic Acid (powder)
0.05% BHT (milled)

A barrier layer was formulated using following materials: 99 wt % ethylcellulose T10 and 1 wt % stearic acid. The stearic acid was dry-blended with the ethylcellulose for approximately 5 minutes.

Compression: The tablets were compressed on a counter press using ¼ ton compression force:

The first 9 sets of tablets comprised:

| | |
|---|---|
| FR1: | 40 mg |
| Barrier Layer Composition: | 30 mg |
| push layer composition: | 100 mg |
| CR 1: | 40 mg |

They were compressed with 3/16″ tooling (LCT).

Tablet sets 10 and 11 comprised:

| Set 10 and 11 | Set 10 | Set 11 |
|---|---|---|
| FR 1 | 67 mg | 100 mg |
| Barrier | 51 mg | 75 mg |
| Push layer | 167 mg | 251 mg |
| CR 1 | 67 mg | 100 mg |

Set 10- 7/32″ tooling (LCT)
Set 11-¼″ tooling (LCT)

For Set 10 and Set 11 the layer weights were adjusted as per control percentages and aspect ratio was maintained similar to control (ie˜2 2)

Tablet set 12 comprised:

| | |
|---|---|
| FR 2: | 40 mg |
| Barrier: | 30 mg |
| Push layer | 100 mg |
| CR 2 | 40 mg |

Next, the tablets were subcoated with HPC: PVP (70:30) in 100% ethanol at 8% solids. Average subcoat weights for tablet sets 1-9 ranged from 10.1 mg to 11.1 mg. Average subcoat weight for tablet set 10 was 11.4 mg. Average subcoat weight for tablet set 11 was 19.3 mg. Average subcoat weight for tablet set 12 was 10.1 mg.

After subcoating, the semi-permeable membrane coating was applied using a Vector® LDCS Hi-coater. The coating solution was made up of 4.95% wt % cellulose acetate, 0.05 wt % PEG 3350, 4.75 wt % purified water, and 90.25 wt % acetone. The coater was set with an exhaust temperature of 25 Deg C., inlet air flow of 30 cfm, pan rpm of 28, pump run % of 36, and solution spray rate of approx. 23 g/min. Average membrane coat weights ranged from 45.74 mg to 48.43 mg.

Next, the coated tablets had orifices drilled by hand with second orifice size as specified above and a first orifice size of 25 mils, and an additional orifice located adjacent to the first orifice, also with a size of 25 mils. The drilled tablets were dried at 45 Deg C and 45 % RH in a humidity controlled oven for 5 days.

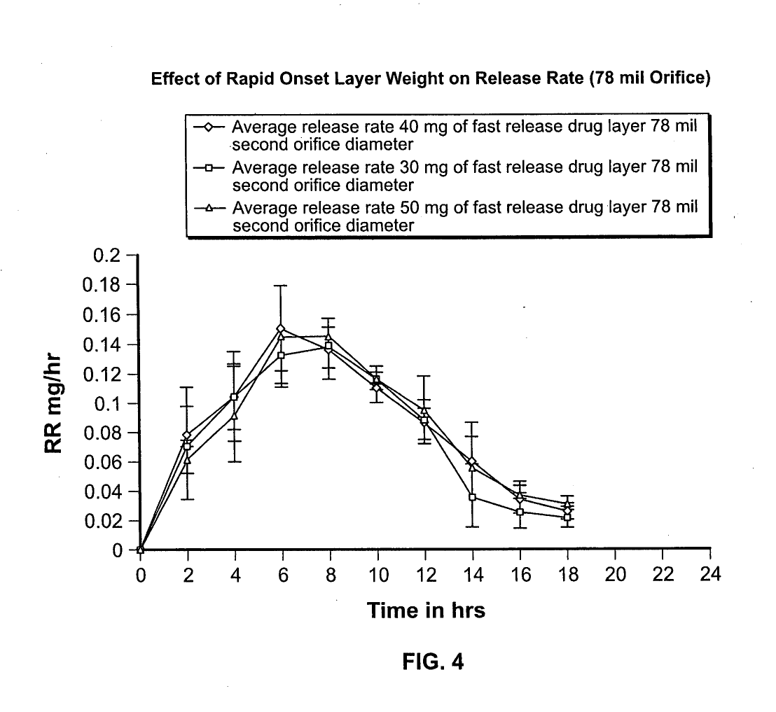

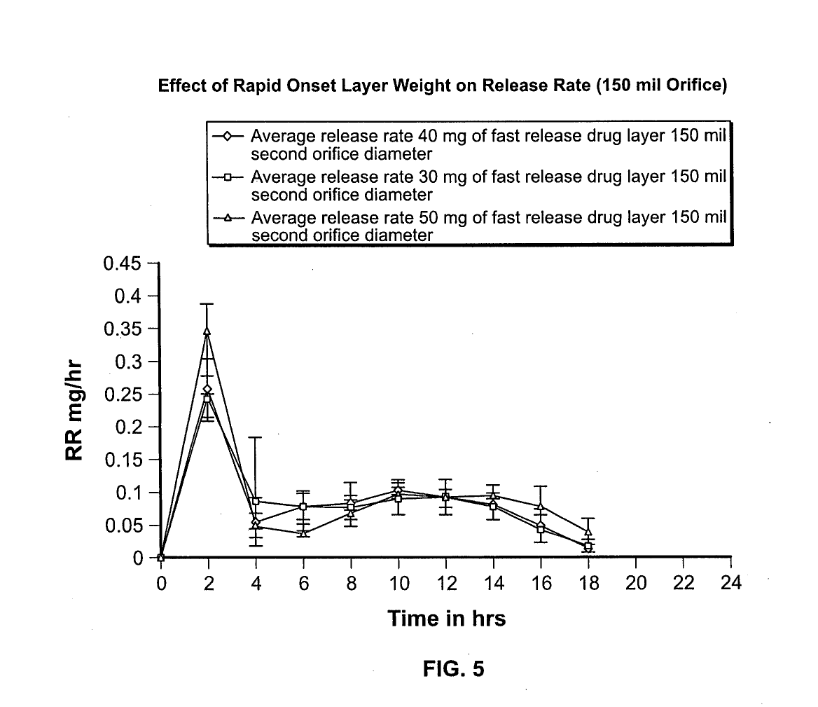

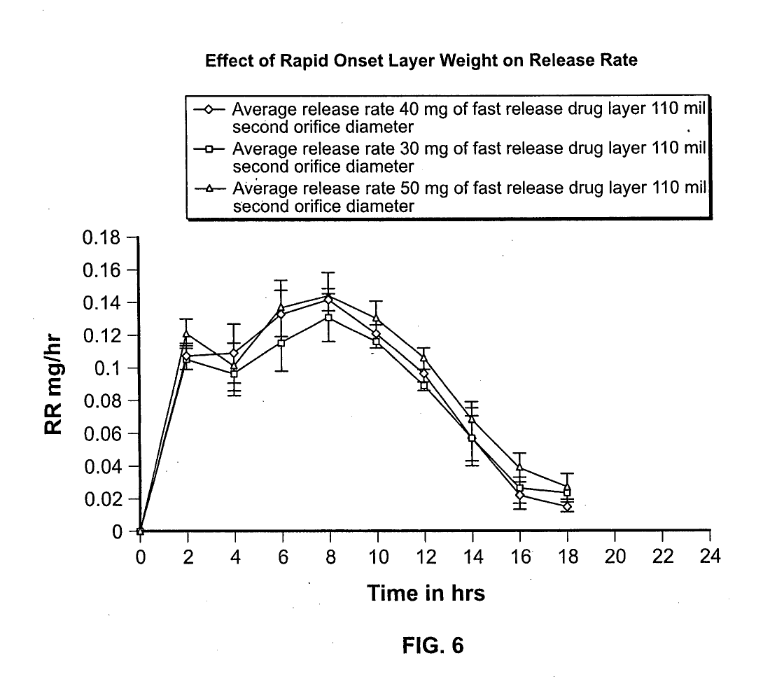

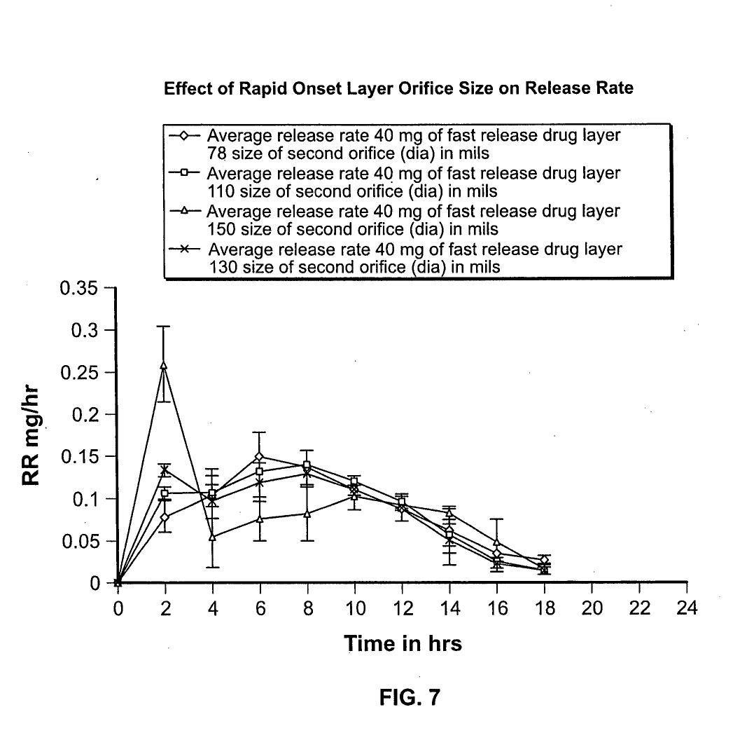

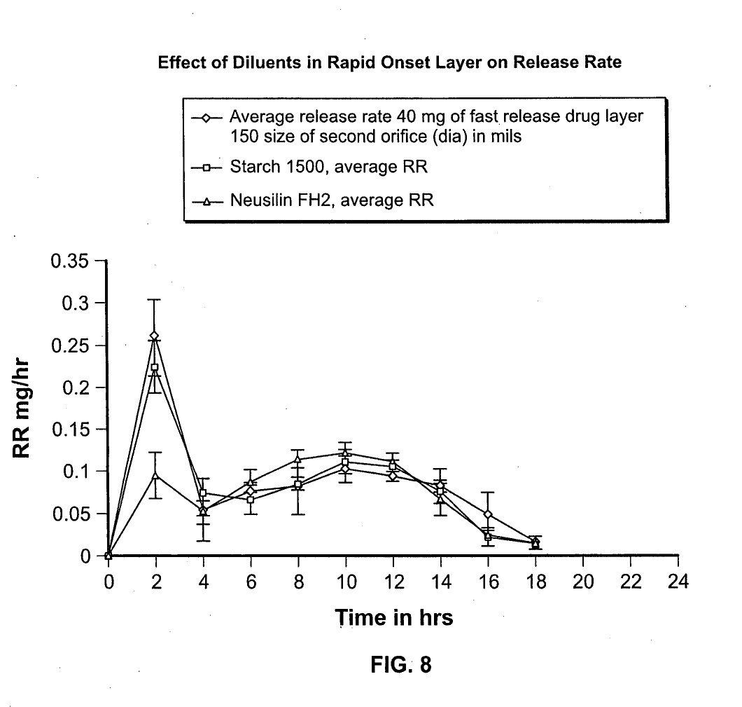

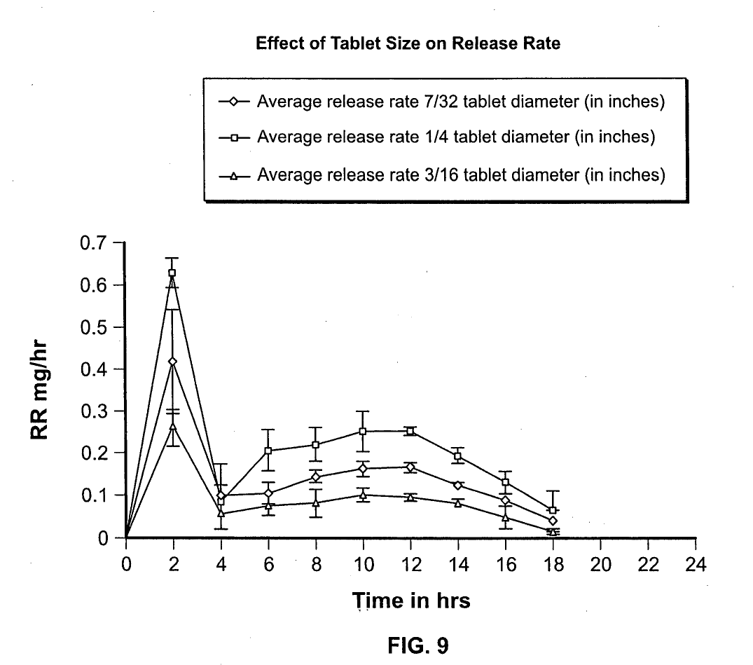

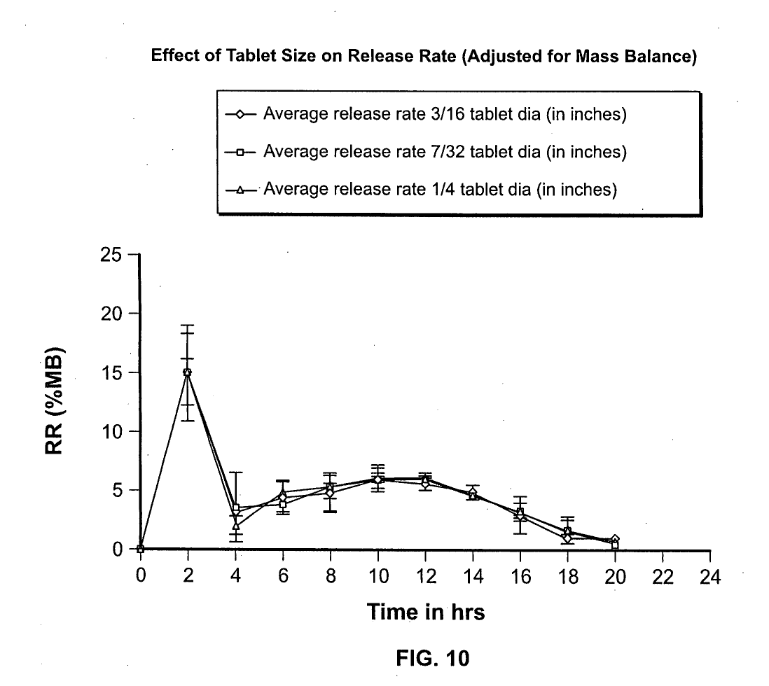

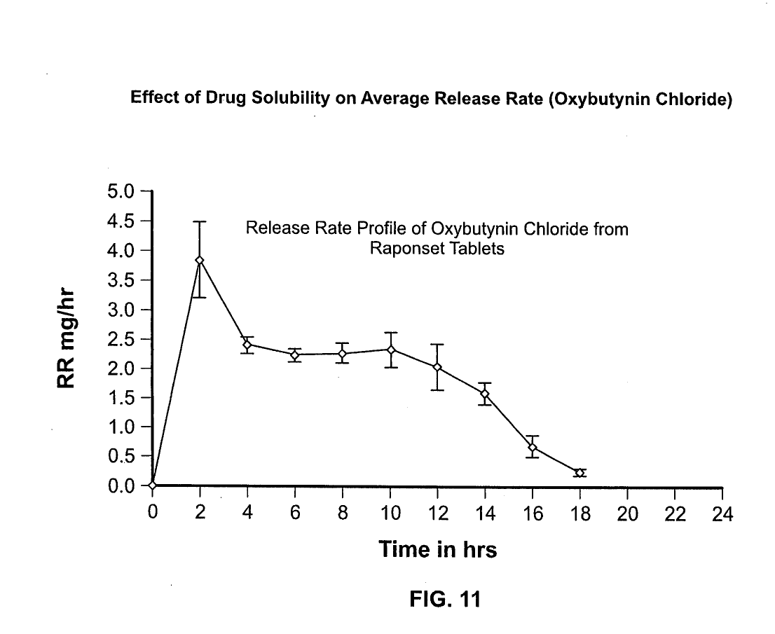

In vitro release performance was assessed in a USP type VII release apparatus. The release medium used was AGF (pH 1.2). The samples were then analyzed using UV absorbance spectrophotometry at a wavelength of 274 nm for paliperidone, or 220 nm and 230 nm for oxybutynin, with 2 hour release time intervals. Release volumes were 50 ml. The results are illustrated in FIGS. 4 though 11.

What is claimed is:

1. An osmotic dosage form comprising:
a semi-permeable membrane;
a first and a second orifice in the semi-permeable membrane located at opposite ends of the semi-permeable membrane;
a controlled release drug layer located adjacent to the first orifice and within the semi-permeable membrane;
a fast release drug layer located adjacent to the second orifice and within the semi-permeable membrane;
a push layer located within the semi-permeable membrane and between the controlled release drug layer and the fast release drug layer;
a barrier layer slidably located between the push layer and the fast release drug layer; and
wherein an area of the second orifice is greater than or equal to about 7800 mil².

2. The osmotic dosage form of claim 1, further comprising one or more additional orifices located in the semi-permeable membrane adjacent to the first orifice.

3. The osmotic dosage form of claim 1, further comprising one or more additional orifices located in the semi-permeable membrane adjacent to the second orifice.

4. The osmotic dosage form of claim 1, wherein the controlled release drug layer comprises an osmagent.

5. The osmotic dosage form of claim 1, wherein the fast release drug layer comprises a disintegrant.

6. The osmotic dosage form of claim 5, wherein the disintegrant comprises croscarmellose sodium or cross-linked polyvinylpyrrolidone.

7. The osmotic dosage form of claim 1, wherein the barrier layer comprises one or more of ethylcellulose, calcium phosphate dibasic, calcium phosphate tribasic, calcium carbonate, polyvinyl acetate, methylcellulose, cellulose (powder), microcrystalline cellulose, polymethyl methacrylate, talc, and/or combinations of the above, and/or any of the above granulated with PVP or HPMC.

8. The osmotic dosage form of claim 1, wherein the barrier layer comprises butyl, propyl, ethyl, and/or methyl paraben, cellulose mono-acetate and/or combinations of these with one or more of ethylcellulose, calcium phosphate dibasic, calcium phosphate tribasic, calcium carbonate, polyvinyl acetate, methylcellulose, cellulose (powder), microcrystalline cellulose, polymethyl methacrylate, talc, and/or combinations of the above, and/or any of the above granulated with PVP or HPMC.

9. The osmotic dosage form of claim 1, wherein the area of the second orifice is greater than or equal to about 10,000 mil².

10. The osmotic dosage form of claim 9, wherein the area of the second orifice is greater than or equal to about 15,000 mil².

11. The osmotic dosage form of claim 10, wherein the area of the second orifice is greater than or equal to about 20,000 mil².

12. The osmotic dosage form of claim 1, wherein the controlled release drug layer and the fast release drug layer comprise different drugs.

13. The osmotic dosage form of claim 1, wherein the controlled release drug layer and the fast release drug layer comprise substantially identical drugs.

14. A method of making an osmotic dosage form comprising:
providing a semi-permeable membrane;
locating a first and a second orifice in the semi-permeable membrane at opposite ends of the semi-permeable membrane;
locating a controlled release drug layer adjacent to the first orifice and within the semi-permeable membrane;
locating a fast release drug layer adjacent to the second orifice and within the semi-permeable membrane;
locating a push layer within the semi-permeable membrane and between the controlled release drug layer and the fast release drug layer;
slidably locating a barrier layer between the push layer and the fast release layer; and
wherein an area of the second orifice is greater than or equal to about 7800 mil².

15. The method of claim 14, further comprising one or more additional orifices located in the semi-permeable membrane adjacent to the first orifice.

16. The method of claim 14, further comprising one or more additional orifices located in the semi-permeable membrane adjacent to the second orifice.

17. The method of claim 14, wherein the controlled release drug layer comprises an osmagent.

18. The method of claim 14, wherein the fast release drug layer comprises a disintegrant.

19. The method of claim 18, wherein the disintegrant comprises croscarmellose sodium or cross-linked polyvinylpyrrolidone.

20. The method of claim 14, wherein the barrier layer comprises one or more of ethylcellulose, calcium phosphate dibasic, calcium phosphate tribasic, calcium carbonate, polyvinyl acetate, methylcellulose, cellulose (powder), microcrystalline cellulose, polymethyl methacrylate, talc, and/or combinations of the above, and/or any of the above granulated with PVP or HPMC.

21. The method of claim 14, wherein the barrier layer comprises butyl, propyl, ethyl, and/or methyl paraben, cellulose mono-acetate and/or combinations of these with one or more of ethylcellulose, calcium phosphate dibasic, calcium phosphate tribasic, calcium carbonate, polyvinyl acetate, methylcellulose, cellulose (powder), microcrystalline cellulose, polymethyl methacrylate, talc, and/or combinations of the above, and/or any of the above granulated with PVP or HPMC.

22. The method of claim 14, wherein the area of the second orifice is greater than or equal to about 10,000 mil².

23. The method of claim 22, wherein the area of the second orifice is greater than or equal to about 15,000 mil².

24. The method of claim 23, wherein the area of the second orifice is greater than or equal to about 20,000 mil².

25. The method of claim 14, wherein the controlled release drug layer and the fast release drug layer comprise different drugs.

26. The method of claim 25, wherein the controlled release drug layer and the fast release drug layer comprise substantially identical drugs.
